SEQUENCE LISTING (5 sequences)

SEQ ID NO: 1 - protein, 414 aa, Artificial
  Fusion protein
MRGSHHHHHHGILRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILGGTGTREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 420 aa, Artificial
  Fusion protein
MRGSHHHHHHGILRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILGGTLVPRGSGTREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 393 aa, Artificial
  Fusion protein
MRGSHHHHHHGILRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILGGTLVPRGSGTVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFGESGQVYFGIIAL

SEQ ID NO: 4 - protein, 385 aa, Artificial
  Fusion protein
MRGSHHHHHHGILRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILGGTVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFGESGQVYFGIIAL

SEQ ID NO: 5 - protein, 717 aa, Artificial
  Fusion protein
MRGSHHHHHHGILRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILGGTLKILNEIASIGSTKQKQAILEKNKDNELLKRVYRLTYSRGLQYYIKKWPKPGIATQSLGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLCSQA

(19) United States
(12) Patent Application Publication
(10) Pub. No.: US 2017/0226489 A1
(43) Pub. Date: Aug. 10, 2017

(54) Solubility and Affinity Tag for Recombinant Protein Expression and Purification

(71) Applicant: Peng Liang, Nashville, TN (US)

(72) Inventor: Peng Liang, Nashville, TN (US)

(21) Appl. No.: 15/015,727

(22) Filed: Feb. 4, 2016

(51) Int. Cl. C12N 9/12; C12N 9/00; C07K 14/525

(52) U.S. Cl. CPC C12N 9/1229; C07K 14/525; C12N 9/93; C12Y 207/04003; C12Y 605/01001; C07K 2319/70

(57) ABSTRACT

Systems and methods of coding for and isolating a fusion protein are provided. The fusion protein may be expressed by a DNA sequence and comprise an adenylate kinase linked by its carboxy-terminus to a biologically active polypeptide or protein. Later processing steps include isolating the biologically active polypeptide or protein via affinity elution of the fusion proteins with substrate analog of adenylate kinase.

### TECHNICAL FIELD

This invention relates to recombinant fusion proteins containing a solubility and affinity tag, genes coding for such proteins, vectors for the expression of these genes, bacteria hosts harboring the expression vectors, and methods for the high level expression of the fusion proteins in soluble form and their subsequent purification.

### BACKGROUND OF THE INVENTION

In the post-genomic era, functional studies of genes rely in part on the expression and characterization of protein products of interest. To this end, the concurrent use of fusion tags with DNA cloning technology has become a routine practice in recombinant protein expression and purification. Although numerous affinity tags have been developed over the years to facilitate the expression and purification of recombinant proteins from Escherichia coli, Glutathione S-transferase (GST-Tag), Maltose-binding protein (MBP-Tag), and 6xHIS-Tag remain the most popular methods of choice due to the commercially available expression vectors and their downstream purification systems.

All tags, whether large or small, can often impede upon the structure and functions of a target protein expressed and may need to be removed during or after purification. Thus, a specific proteolytic cleavage site is often introduced between the tag and the protein of interest to be expressed on an expression vector. Among many site specific proteases used for the cleavage of the tags off target proteins, thrombin is the most widely used due to its high target sequence-specificity and rarity found in natural proteins.

Despite the wide-spread use of these tailor-made expression vectors and purification strategies, frustrations often occur when a target protein is expressed either at low level, or as insoluble inclusion bodies. Although His-Tag may allow purification of insoluble proteins after complete unfolding with a denaturant, the yield of refolding and full recovery of biological functions of a recombinant protein may be less predictable. In addition, depending on the level of expression, the yield and purity of a recombinant protein from a single affinity column can be far from perfect due to endogenous host cell proteins bound to the columns.

### SUMMARY OF THE INVENTION

Adenylate Kinase (AK) catalyzes the enzymatic conversion of AMP to ADP using ATP: Mg²⁺ATP+AMP⇄Mg²⁺ADP+ADP, and is an essential enzyme in all living organisms). Previous work from our laboratory as well as by others showed that AK from Escherichia coli could be not only expressed at high level in soluble and active form in bacteria, but also easily purified to near homogeneity in a single step involving the affinity elution of the enzyme bound to Blue-Sepharose beads with its transitional state substrate analog, P1,P5-Di (adenosine-5′) pentaphosphate (Ap5A). We envisioned that AK, when used as a protein fusion tag, may not only aid the soluble expression of a recombinant protein which could be easily verified by a sensitive enzymatic AK reaction, but also allow the recombinant protein to be purified in one step to a level unattainable by any previously published affinity tags. Here we demonstrate the proof of concept in an effort to express TNF family of cytokines as AK fusion proteins. Moreover, by introducing a His-Tag at the N-terminus and a thrombin cleavage site at the C terminus of AK, the method we dubbed AK-TAG can also allow the expression and recovery of a fully functional native recombinant protein in high yield and purity via dual affinity purification steps.

### BRIEF DESCRIPTION OF THE DRAWINGS

Table 1. Purification Yield of AK-TNFα and AK-T-TNFα from 100 ml of Bacterial culture

Table 2. Bioactivity of AK-TNFα and TNFα

FIG. 1A. pAK-TAG expression vector and high-level expression of recombinant AK fusion proteins in soluble form. Schematic representation of the pAK-TAG vector.

FIG. 1B pAK-TAG expression vector and high-level expression of recombinant AK fusion proteins in soluble form. 15% SDS-PAGE analysis of the expression of AK-TNFα, AK-TRAIL, and AK-T4 DNA ligase.

FIG. 2. One step purification of AK-TNFα via Ap5A affinity elution off Blue-Sepharose chromatography. M: Protein-molecular-weight size markers. Lane 1: Cleared soluble bacterial cell lysate. Lane 2: Flow-through fraction from Blue-Sepharose column. Lane 3: Ap5A elution of AK-TNFα from Blue-Sepharose

FIG. 3A. Construction of pAK-TAG-T expression vector with a thrombin cleavage site and the expression and purification of the native TNFα. Schematic representation of pAK-TAG-T expression vector.

FIG. 3B. Construction of pAK-TAG-T expression vector with a thrombin cleavage site and the expression and purification of the native TNFα. 15% SDS-PAGE analysis of the expression of AK-T-TNFα.

FIG. 3C. Construction of pAK-TAG-T expression vector with a thrombin cleavage site and the expression and purification of the native TNFα. SDS-PAGE analysis the purification of the native TNFα. AK-T-TNFα fusion protein was first purified via Ap5A affinity elution off a Blue-Sepharose column followed by purification of the native TNFα off a Ni-NTA column upon thrombin digestion. M: Protein-molecular-weight size markers. Lane 1: Cleared soluble bacterial cell lysate. Lane 2: Flow-through fraction from Blue-Sepharose column. Lane 3: Ap5A Elution from Blue-Sepharose. Lane 4: Ni-NTA column and thrombin release of the native TNFα. Lane 5: Imidazole elution of AK from the Ni-NTA column.

FIG. 3D. Construction of pAK-TAG-T expression vector with a thrombin cleavage site and the expression and purification of the native TNFα. Estimate of the level of AK fusion protein expression by AK enzyme activity.

FIG. 4A. Characterization of the purified AK-TNFα and native TNFα. SEC-HPLC analysis of the purity and estimate the molecular-weight of AK-TNFα and TNFα.

FIG. 4B. Characterization of the purified AK-TNFα and native TNFα. Analysis of the biological function (L929 assay) and determination the IC50 of AK-TNFα and native TNFα in comparison to a commercial source of TNFα (R&D Systems).

FIG. 5. 15% SDS-PAGE analysis of the expression of AK-T-4-1BBL. AK-T-4-1BBL fusion protein was first purified via Ap5A affinity elution off a Blue-Sepharose column followed by purification of the native 4-1BBL off a Ni-NTA column upon in column thrombin digestion. MW: Protein-molecular-weight size markers in Kda. Lane 1: Cleared soluble bacterial cell lysate. Lane 2: Flow-through fraction from Blue-Sepharose column. Lane 3: Ap5A elution from Blue-Sepharose. Lane 4: after loading to a Ni-NTA column, in-column thrombin cleavage and release of the native 4-1BBL. Lane 5: Imidazole elution of AK from the Ni-NTA column.

### DESCRIPTION OF SEQUENCE LISTING

SEQ. ID NO. 1 Shows the amino acid sequence of AK and human TNFα fusion protein (AK-TNFα) without protease cleavage site in the linker region.

SEQ. ID NO. 2 Shows the amino acid sequence of AK and human TNFα fusion protein (AK-T-TNFα) with a thrombin protease cleavage site (T) located in the linker region.

SEQ. ID NO. 3 Shows the amino acid sequence of AK and human 4-1BBL fusion protein (AK-4-1BBL) without protease cleavage site in the linker region.

SEQ. ID NO. 4 Shows the amino acid sequence of AK and human 4-1BBL fusion protein (AK-T-4-1BBL) with a thrombin protease cleavage site (T) located in the linker region.

SEQ. ID NO. 5 Shows the amino acid sequence of AK and T4 DNA Ligase fusion protein (AK-T4 DNA Ligase) without protease cleavage site in the linker region.

### DESCRIPTION OF THE INVENTION

The detailed description set forth below in connection with the appended drawings and sequence listing is intended as a description of presently preferred embodiments of the invention and does not represent the only forms in which the present invention may be constructed and/or utilized. The description sets forth the functions and the sequence of steps for constructing and operating the invention in connection with the illustrated embodiments.

### Material and Methods

### Chemicals

T4 DNA ligase, Taq polymerase, and restriction enzymes were obtained from Takara. Isopropyl-beta-D-thiogalactopyranoside (IPTG) was purchased from Merck. 3-(4,5-dimethyl-2-thiazolyl)-2,5-diphenyl-2-H-tetrazolium bromide(MTT), Fetal Bovine Serum (FBS) , P1, P5-di(adenosine-5′) pentaphosphate (Ap5A), glucose, ADP, NADP+, hexokinase and glucose-6-phosphate dehydrogenase were from Sigma. Thrombin (Restriction Grade) was obtained from Millipore.

### Strains and Plasmids

pQE32 (Qiagen) was propagated in Escherichia coli XL1-Blue (Qiagen). pAK-T4 and pKILLIN vectors were described recently by our laboratory (Use of Adenylate Kinase as a Solubility Tag for High Level Expression of T4 DNA Ligase in Escherichia coli, Liu et al., Protein Expression and Purification 109 (2015) 79-85, Feb. 17, 2015, hereinafter referred to as Liu et al. 2015, the entire contents of which is incorporated herein by reference).

### Plasmid DNA Constructs

The creation of the pAK-TAG expression vector was initially (within 1 year before the filing of the current invention) described by our own laboratory in the expression of AK-T4 DNA ligase fusion protein (Liu et al. 2015). Thrombin cleavage site was then inserted into pAK-TAG at the end of the AK coding region before the multiple cloning sites using a linker composed of L-thrombin (5′-GCTGGTGCCGCGTGGCAGCGGTAC-3′) and R-thrombin (5′-CGCTGCCACGCGGCACCAGCGACG-3′) primers. The resulting vector was designated as pAK-TAG-T. For the expression of AK-TNFα fusion proteins, DNA fragment encoding the mature human TNFα was amplified by PCR and cloned into pAK-TAG and pAK-TAG-T vectors at Kpn I and Hindlll sites. All plasmid constructs were verified by DNA sequence analysis.

### Enzyme Assay for Adenylate Kinase (AK)

AK enzyme assay was carried out. The reaction mixture (50 μl) consists of 10 mM glucose, 2 mM ADP, 0.5 mM NADP+, 1.2 units of hexokinase and 0.6 units of glucose-6-phosphate dehydrogenase (Sigma). The AK activity was calculated by the rate of increase in absorbance at 340 nm. The formation of 1 μmol of ATP in one minute is defined as one unit of AK enzyme activity. Protein concentration was measured by BCA assay kit (Pierce), according to the manufacturer's instructions.

Expression of Recombinant pAK-TNFα and pAK-T-TNFα in Escherichia coli

Cell growth and induction of expression was carried out using processes known in the art (Liu et al. 2015). Briefly, bacteria cells were grown in LB medium at 37° C. until reaching 0.6 at OD600, 1 mM IPTG was then added to induce the protein expression for 10 h at 30° C. After induction, cells were harvested by centrifugation and protein extracts were prepared by sonication in extraction buffer as in a manner known in the art.

### Quantification of AK Fusion Protein Expression by AK Activity

The level of a soluble AK fusion protein expression was estimated by the fold of increase in the specific activity of AK over that of the host cell XL1-blue containing pQE32.

### Purification of AK-TNFα and AK-T-TNFα by Blue-Sepharose Column

After IPTG induction, the cells were collected by centrifugation at 4000 g for 20 min at 4° C. The cell pellet was re-suspended in 20 ml of buffer A (20 mM Tris-HCl, pH 7.4, 1 mM MgCl₂) and then lysed by sonication. Cell debris was removed by centrifugation at 16900 g for 20 min at 4° C. Protein purification was carried out using an AKTA-Prime purification system (GE Healthcare). About 80 mg of cleared cell lysate was used as a starting material and passed through a 1 ml HiTrap Blue HP (Blue-Sepharose) column (GE Healthcare) pre-equilibrated with 20 Column volumes (CVs) of buffer A. After sample loading, 10 CVs buffer A was added to wash off weakly bound proteins until OD280 detection declined to near baseline. The AK-TNFα and AK-T-TNFα fusion protein was eluted with a 20 CVs buffer B (20 mM Tris-HCl, pH 7.4, 50 μM Ap5A, 1 mM MgCl₂). The flow rate was maintained at 1 ml/min throughout the purification according to known methods.

### Purification of Native Human TNFα by Ni-NTA Column

AK-T-TNFα eluted from Blue-Sepharose column was passed through a Ni-NTA column (GE Healthcare) which was pre-equilibrated with 15 CVs of buffer C (20 mM Tris-HCl, pH 7.4, 500 mM NaCl, 5 mM imidazole). After washing off weakly bound contaminating proteins with 15 CVs of buffer C until OD280 declined to near the baseline, another 15 CVs of buffer D (20 mM Tris-HCl, pH 8.4, 150 mM NaCl, 2.5 mM CaCl₂) was passed through the column. In-column thrombin digestion was initiated after passing 1.5 ml thrombin (9U in buffer D) and kept at the room temperature for 20 h. The column was washed with 10 CVs of buffer D followed by elution of the native human TNF with 10 CVs of buffer C. The His-tagged AK was finally eluted with a 15 CVs linear-gradient of 5-500 mM imidazole in buffer E (20 mM Tris-HCl, pH 7.4, 500 mM NaCl, 500 mM imidazole).

### SEC-HPLC Analysis of Protein Purity and Molecular-Weight

The purity of purified AK-TNFα and TNFα was evaluated on an Agilent 1260 HPLC using Size-Exclusion Chromatography (SEC-HPLC) with an analytic TSK G3000SWXL column (Tosoh). Phosphate Buffered Saline (PBS) was used as the mobile phase with OD280 nm detection over a 20 min period at a flow rate of 1 ml/min. The protein molecular weight was estimated based on retention time using the protein size standard (BioRad).

### Cytotoxicity Assay

The biological activity of TNFα was measured in vitro according to established method (Shiau et al. 2001). Briefly 1×104 cells of L929 cells (ATCC) was seeded into each well of a 96-well cell plate with RPMI-1640 (Hyclone) containing 10% FBS. After 24 h, actinomycin D was added to the medium at 1 μg/ml together with various concentrations of either TNFα (R&D), native TNFα or AK-TNFα. After 18 hours of cell culture at 37° C., 0.5 mg/ml MTT was added to each well followed by a 4 h of incubation at 37° C. Then the media in the wells were carefully discarded and 100 μl of dimethylsulfoxide (DMSO) was added into each well to dissolve Formazan crystal. The absorbance at 490 nm was recorded.

### Results

### High-Level Expression of Recombinant AK Fusion Proteins in Soluble Form

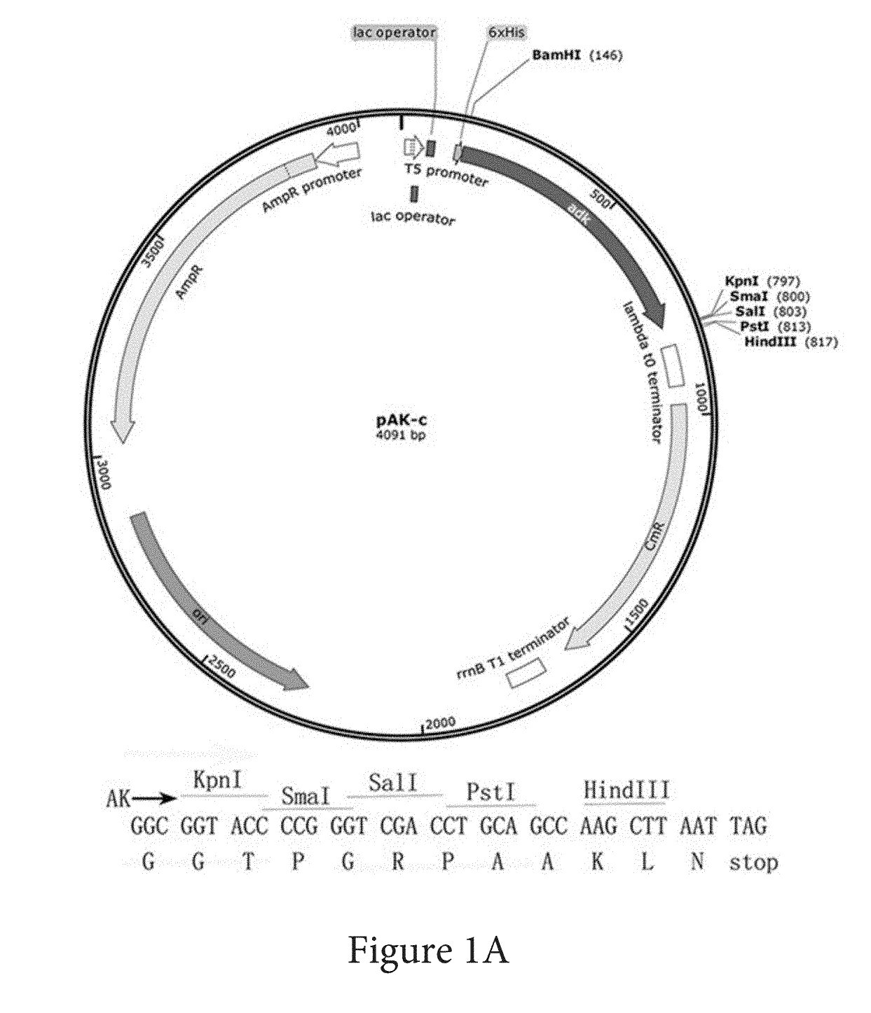

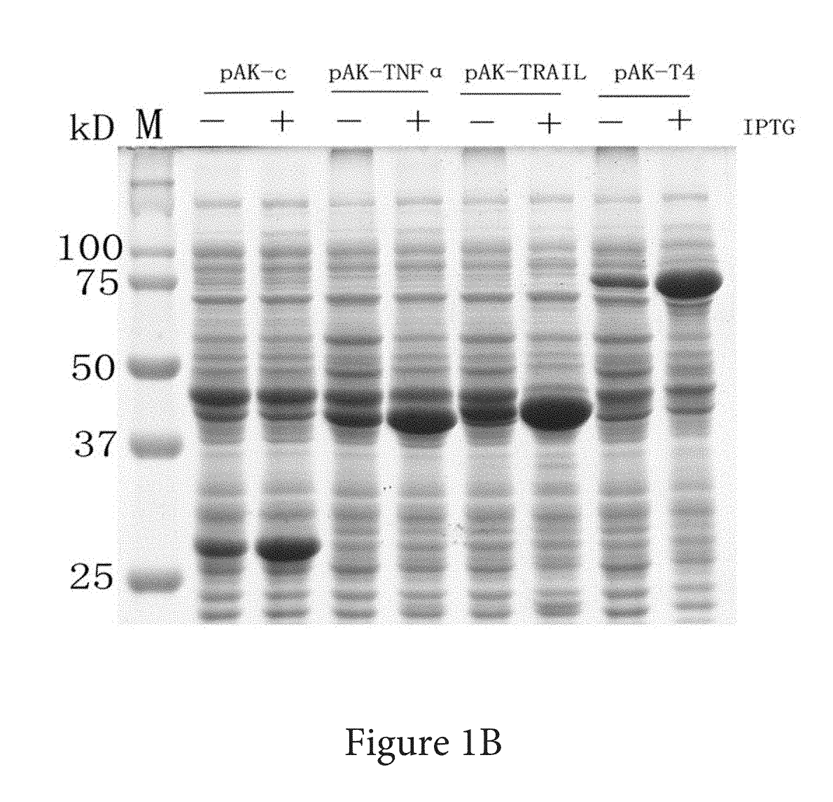

In a preferred embodiment, We have demonstrated the proof of principle for the current invention by describing the expression of DNA ligase from bacteria phage T4 in soluble form as an AK fusion protein using pAK-TAG cloning vector we developed (Liu et al. 2015) (FIG. 1a). pAK-TAG expression vector contains multiple cloning sites at the carboxy-terminus (C-terminus) of AK followed by a stop codon for convenient in-frame fusion of any recombinant protein. Under the control of a strong T5 promoter and the lac operator, the N-terminal His-tagged AK alone or any AK fusion proteins may be expressed at high level upon IPTG induction (FIG. 1a) such as AK-T4 DNA ligase.

In another preferred embodiment, to further demonstrate that the same goal can be attained for secreted proteins from mammalian cells, we have constructed several AK fusion proteins from human TNF family of cytokines. These fusion proteins, including AK-TNFα and AK-Trail as shown in FIG. 1b, like T4 DNA ligase, were also expressed at high level in soluble forms upon IPTG induction. AK alone has a molecular weight at 27 kDa and both monomeric human TNFα and Trail have molecular weights around 16-17 kDa. The predicted molecular weights for AK-TNFα and AK-Trail thus would be around 43-44 kDa, which were consistent with our data presented (FIG. 1b).

One Step Purification of the AK-TNFα Fusion Protein by Blue-Sepharose with Ap5A Affinity Elution

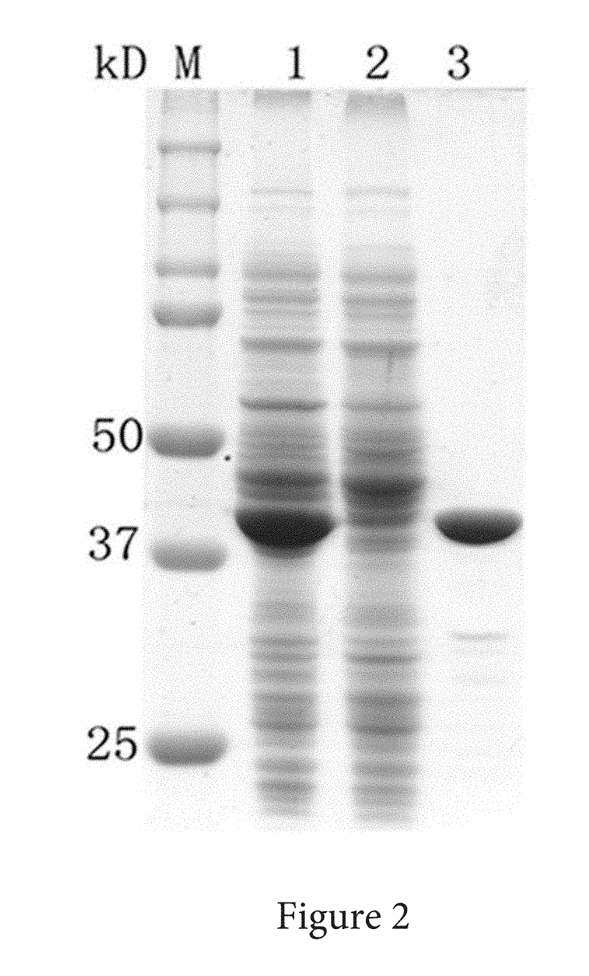

The original concept for using AK as an affinity tag was intended to take the advantage of its high-level bacterial expression, sensitive detection by enzyme assay and easy purification. However, the first AK fusion protein (AK-T4 DNA ligase) we expressed failed to come off the Blue-Sepharose column upon Ap5A elution due to the ability of T4 DNA ligase to bind to nucleotide-like dye moiety from the chromatographic matrix on its own. We next chose proteins from mammalian cell origins that are known not to pertain nucleotide or DNA binding properties, which are responsible for Blue-Sepharose binding. We showed that the AK-TNFα fusion protein behaved similarly to AK itself and was able to bind to Blue-Sepharose beads with little flow through. Upon complete washing off of any unbound proteins, AK-TNFα was efficiently and specifically eluted off the column by Ap5A with little contaminating proteins (FIG. 2).

Construction of pAK-TAG-T Expression Vector with Thrombin Cleavage Site

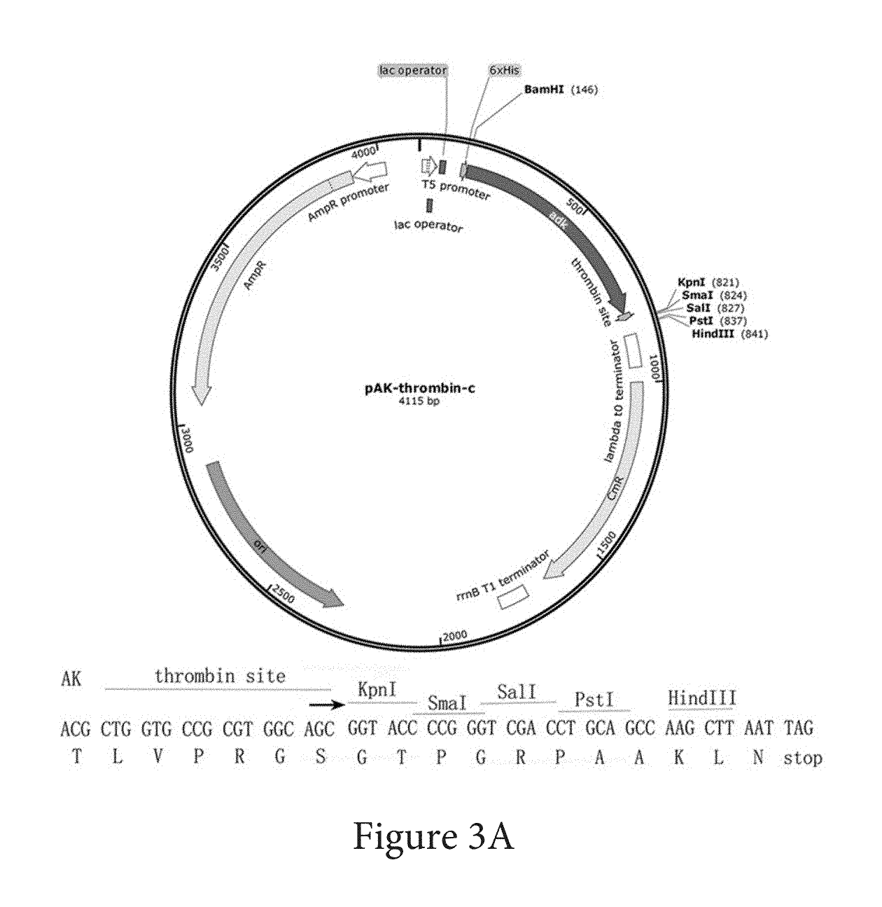

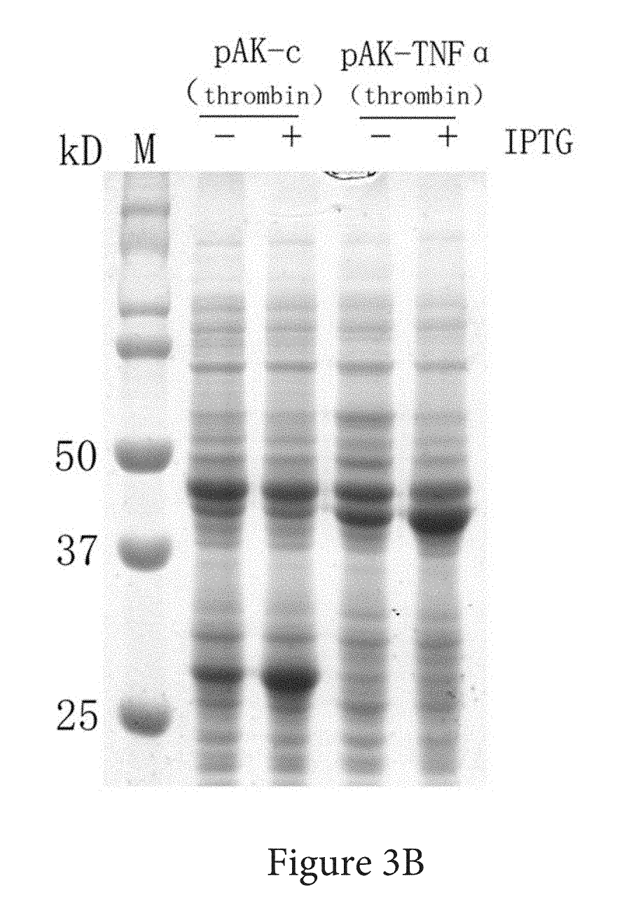

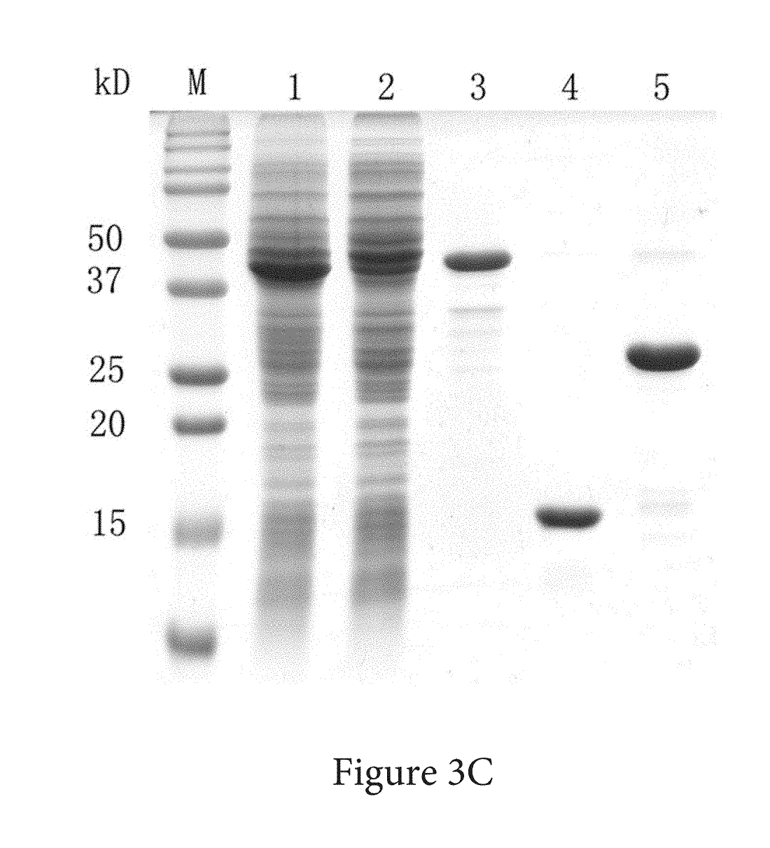

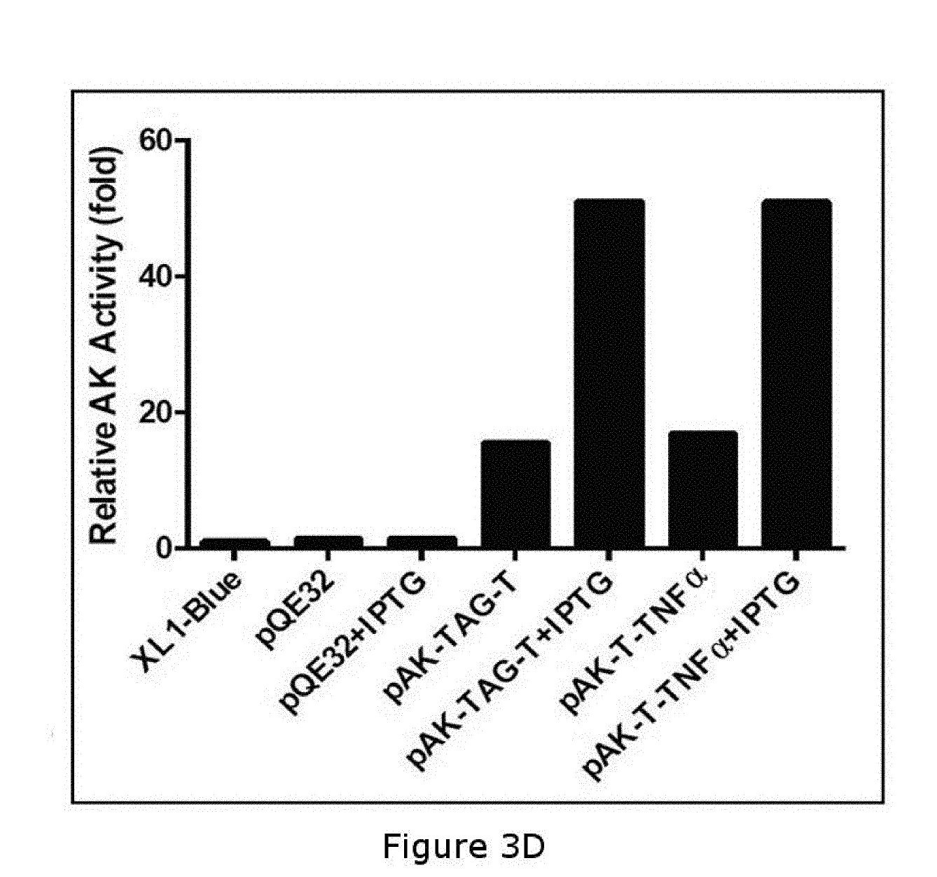

In yet another preferred embodiment, to ensure that a native recombinant protein could be recovered from its AK-fusion form, a thrombin cleavage site was introduced in-frame into the pAK-TAG expression vector before the multiple cloning sites. The resulting expression vector was designated as pAK-TAG-T (FIG. 3a).

### High-Level Expression of AK-T-TNFα and Estimation of its Expression via AK Activity

When human TNFα was cloned into pAK-TAG-T vector and transformed into XL1-blue, the resulting AK-T-TNFα fusion protein with a thrombin cleavage site in-between was expressed at high levels in soluble form upon IPTG induction, in comparison to AK expression from the pAK-TAG-T vector alone (FIG. 3b). The level of AK-T-TNFα was easily measured via AK activity. Based on the increase in the specific activity of AK, AK-T-TNFα expression level was estimated to be over 50 times of endogenous AK from the host cells (FIG. 3D). Due to the high copy number of the pAK-TAG-T vector and strong T5 promoter, there were leaky expression of AK-T and AK-T fusion proteins without the inducer.

The one step purifications of AK-TNFα and AK-T-TNFα were easily monitored by the AK activity of the fusion proteins. The yield of recovery was estimated to be around 50% with over 7-fold purification of the fusion proteins based on increase in specific activity of AK (Table 1).

### Two Step Purification of Native Human TNFα

Like described above, the AK-T-TNFα fusion protein with a thrombin cleavage site was first purified to near homogeneity via Blue-Sepharose with Ap5A elution (FIG. 3C). To release the native TNFα from AK, the purified AK-T-TNFα fusion protein was then loaded onto a Ni-NTA column. Because the AK was N-terminal His-tagged, the AK-T-TNFα was captured by the Ni-NTA. Then thrombin was added to allow in-column cleavage of the fusion protein overnight. The native human TNFα was then released from the Ni-NTA column with the elution buffer with low concentration of imidazole, while the bound AK moiety was later eluted with a linear-gradient of 5-500 mM imidazole (FIG. 3C).

### Characterization of Purified Human TNFα and AK-TNFα

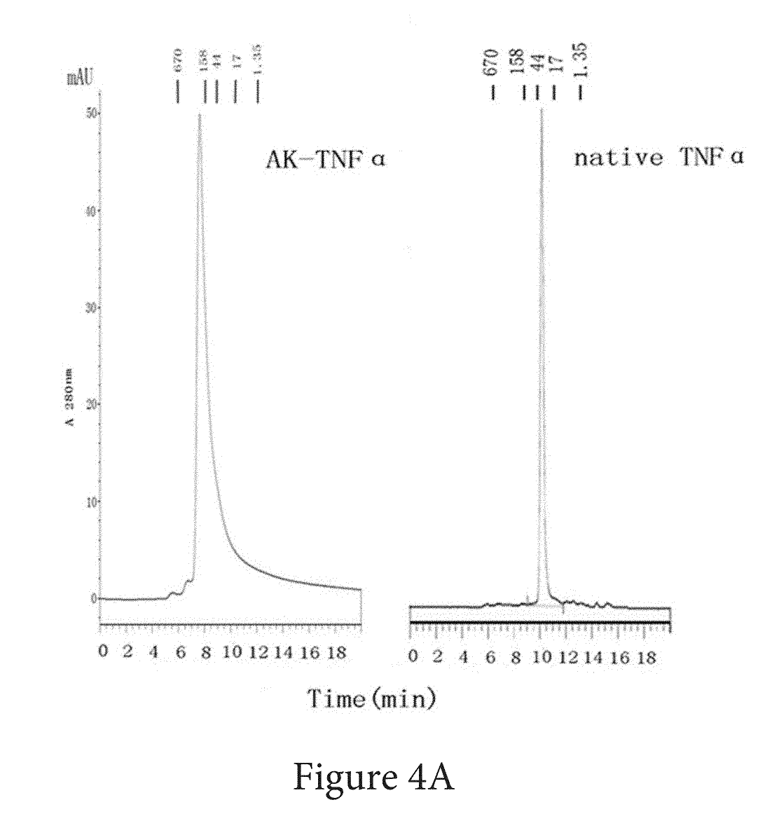

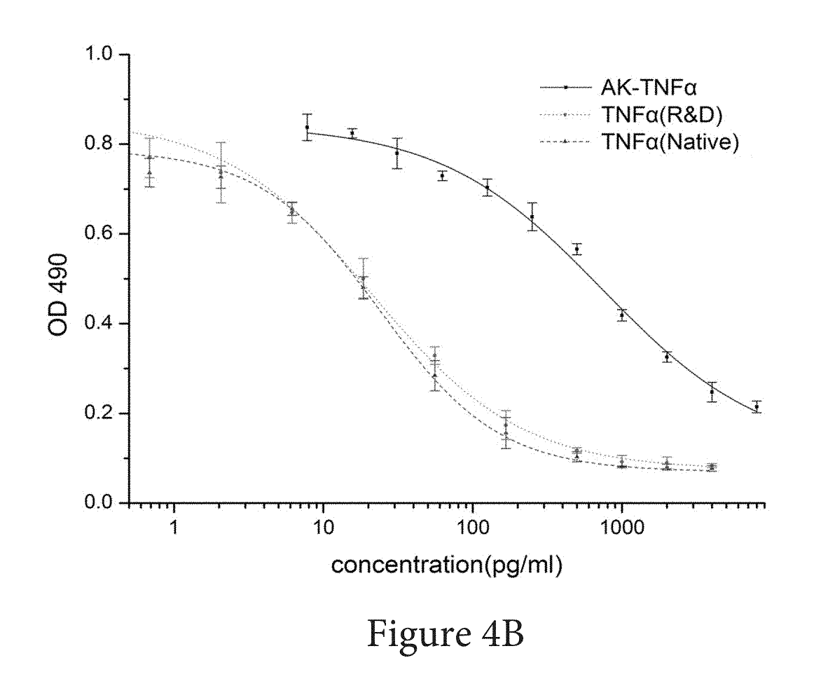

TNFα family of cytokines are all trimeric in structure which is required for their full biological functions. To determine the conformation of AK-TNFα fusion protein and the native TNFα purified with our AK-TAG method, we analyzed them on a size-exclusion HPLC (SEC-HPLC). Both purified AK-TNFα and the native TNFα showed as single peaks with estimated molecular weight consistent with being trimers (FIG. 4a). Functionally, the AK-TNFα fusion protein not only had the AK activity but also retained TNFα biological function as determined by its cytotoxicity to L929 cells (Shiau et al. 2001)(FIG. 4b). However, its potency was over 13 times less than that of the native TNFα upon being released from thrombin cleavage (FIG. 4b). The native TNFα generated with our AK-TAG method exhibited the comparable potency in IC50 in L929 bioassay to TNFα from R&D systems (Table 2).

### High-Level Expression of AK-T-4-1BBL and Two Step Purification of Native Human 4-1BBL

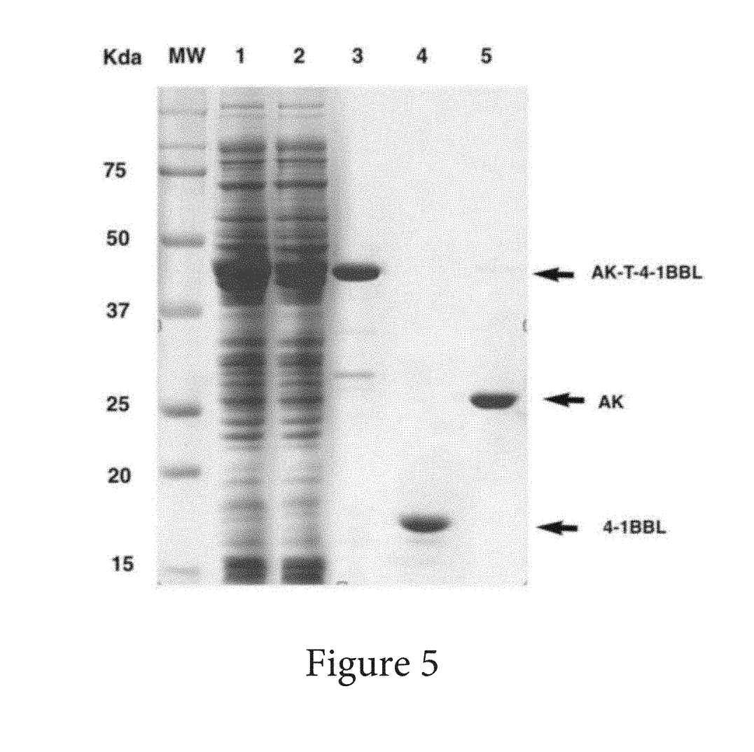

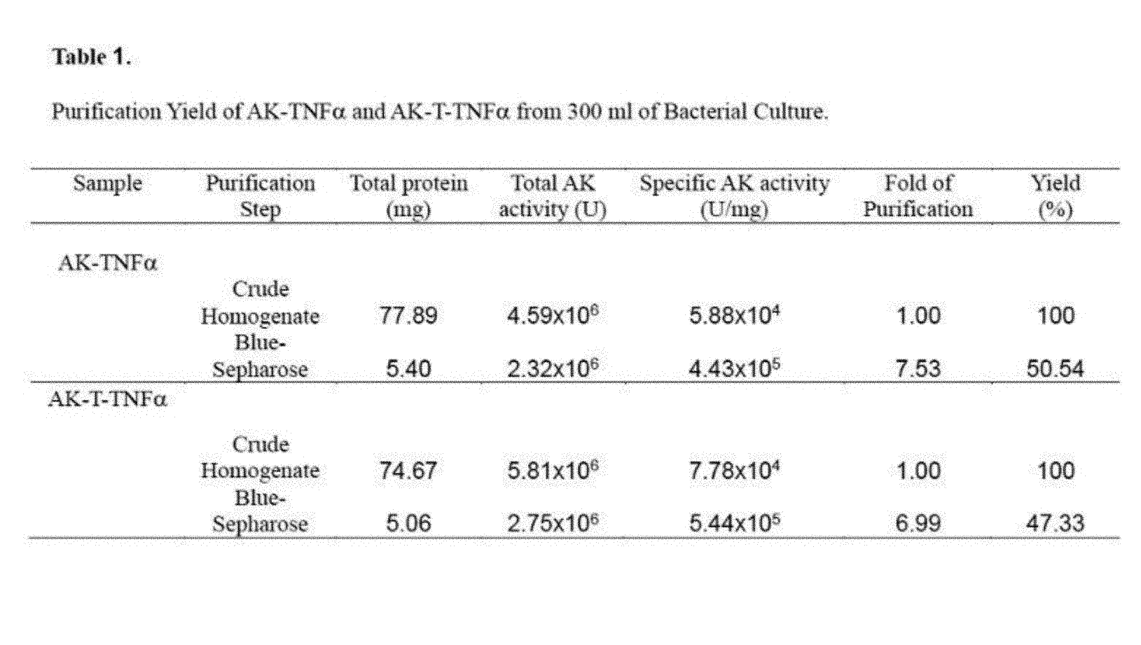

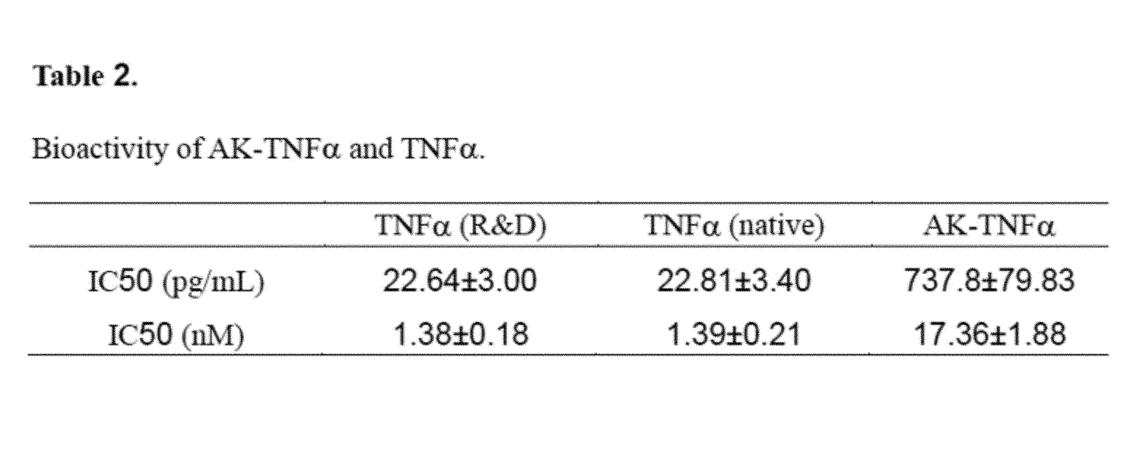

In another preferred embodiment, we cloned another member of TNF family of cytokines, human 4-1BBL into either pAK-TAG or pAK-TAG-T vectors and transformed them into XL1-blue, the resulting AK-4-1BBL and AK-T-4-1BBL fusion proteins were expressed at high level in soluble forms upon IPTG induction, (FIG. 5). The level of AK-T-4-1BBL was easily measured via AK activity. Based on the increase in the specific activity of AK, AK-T-4-1BBL expression level was estimated to be over 50 times over that of endogenous AK from the host cells.

As in the case of AK-T-TNFα fusion protein with a thrombin cleavage site, the AK-T-4-1BBL fusion protein was first purified to near homogeneity via Blue-Sepharose with Ap5A elution (FIG. 5). To release the native 4-1BBL from AK, the purified AK-T-4-1BBL fusion protein was then loaded onto a Ni-NTA column. Because the AK was N-terminal His-tagged, the AK-T-4-1BBL was captured by the Ni-NTA. Then thrombin was added to the buffer to allow in-column cleavage of the fusion protein overnight. The native human 4-1BBL was then released from the Ni-NTA column with the elution buffer with low concentration of imidazole, while the bound AK moiety was later eluted with a linear-gradient of 5-500 mM imidazole (FIG. 5).

### Discussion

Although the concept of using E. coli adenylate kinase as both a solubility and affinity tag for high level bacterial expression and purification of combination proteins is appealing, its reduction to practice was not fool-proof for several. It is not clear a bacterial enzyme can be fused to a foreign protein and still maintains its native conformation and biological function. In fact, our initial effort in trying to fuse a foreign protein to the N-terminus of AK failed to lead the expression of the fusion protein, likely due to mis-folded conformation. In above preferred embodiments, we demonstrated that multiple foreign proteins including that from mammalian origins could be expressed at high level in soluble forms when they were fused to the C-terminus of AK. The functional dye ligand from Blue-Sepharose is Cibacron Blue F3G-A. Some proteins and enzymes such as AK interact bio-specifically with the dye due to its structural similarity with nucleotide cofactors or substrates, while others, such as albumin and interferon, bind in a less specific manner by electrostatic and/or hydrophobic interactions with the aromatic anionic ligands We showed that the AK-TNFα fusion protein behaved similarly to AK alone and could be efficiently purified to near homogeneity in a single chromatographic step using Blue-Sepharose via Ap5A affinity elution. Ap5A, which is a transition state substrate analog of AK, binds to E. coli AK with high affinity and enabled the complete elution of AK-TNFα at M range. This is in contrast to eluents used for other affinity tags, which often require eluents several orders of magnitude higher in concentration such as imidazole (for His-tag). Such a high specificity of Ap5A to AK ensures an unparalleled purity achievable with AK-TAG.

As noted above, some target proteins, unlike TNFα may bind to Blue-Sepharose on their own in a less specific manner via electrostatic and/or hydrophobic interactions with the aromatic anionic ligand. In these cases, such AK fusion proteins may not be eluted by Ap5A alone and require less specific elution with a proper salt concentration such as in the case of AK-T4 DNA ligase fusion protein. Although salt elution is less specific, remaining impurity may be easily and completely removed via a subsequent Ni-NTA column with imidazole elution, by taking the advantage of a 6xHis-tag engineered at the N-terminus of AK.

Another advantage of using AK as both a solubility and affinity tag for recombinant protein expression and purification is its highly active enzyme activity and a relative easy assay. The extent of soluble expression of a recombinant AK fusion protein can be quickly and quantitatively assessed by AK enzyme activity assay in comparison with that of the host cells, which can be achieved within minutes, in contrast to the lengthy SDS-PAGE analysis. Also, an AK fusion protein may be monitored in near real time by the AK activity during the purification, which can allow yield of recovery to be easily for each purification step.

For any affinity tag used for a recombinant protein production, it is often desirable to have the option of having the tag being removed in order to obtain a native target protein with a full biological function. To this end, we introduced a thrombin cleavage site at the C-terminus of AK to obtain the pAK-TAG-T expression vector. This allows us to purify the native TNFα protein to homogeneity with ease by a Blue-Sepharose column via Ap5A elution followed by in-gel thrombin release on a Ni-NTA column, while AK-TAG remains bound to the matrix though a 6xHis-tag. Thus, AK-TAG is a highly streamlined method for high-level soluble expression and fine purification of recombinant proteins. It complements other expression vectors with different affinity tags and is particularly useful when high yield and purity of a native recombinant protein is desired.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A DNA sequence coding for a fusion protein comprising an adenylate kinase linked by its carboxy-terminus to a biologically active polypeptide or protein.

2. The DNA sequence of claim 1 wherein the adenylate kinase is from E. coli.

3. A recombinant expression vector comprising a promoter located upstream of the DNA sequence coding for the fusion protein of claim 1, wherein the recombinant expression vector is capable of directing expression of the DNA sequence in a compatible unicellular host organism.

4. A unicellular organism containing the recombinant expression vector comprising the promoter located upstream of the DNA sequence coding for the fusion protein of claim 3, wherein the unicellular organism is capable of expressing the DNA sequence.

5. The unicellular organism of claim 4 which is E. coli.

6. A process for producing a fusion protein comprising:
(a) culturing a unicellular organism containing a recombinant expression vector comprising a promoter located upstream of a DNA sequence coding for a fusion protein comprising an adenylate kinase linked by its carboxy-terminus to a biologically active polypeptide or protein under conditions in which the fusion protein is produced; and
(b) isolating the fusion protein from the cell culture.

7. The process of claim 6 in which the fusion protein is isolated via the steps of:
(a) contacting a cell lysate or protein extract of the unicellular organism cell culture containing the fusion with a resin to which adenylate kinase binds through its enzyme activate site to form a resin-fusion protein complex;
(b) washing the resin-fusion protein complex with a buffer to remove unbound proteins; and
(c) eluting the bound fusion protein from the washed resin-fusion protein complex with a buffer containing an appropriate amount of a substrate or substrate analog of adenylate kinase.

8. The process of claim 7 in which the resin contains covalently linked Cibacron blue 3GA dye molecules.

9. The process of claim 7 in which the substrate analog is P1,P5-Di (adenosine-5′) pentaphosphate (Ap5A).

10. The process of claim 6 in which the link between the adenylate kinase and the biologically active polypeptide or protein of the isolated fusion protein cannot be cleaved with a protease to separate the biologically active polypeptide or protein from an adenylate kinase.

11. The process of claim 6 in which the link between the adenylate kinase and the biologically active polypeptide or protein of the isolated fusion protein can be cleaved with a protease to separate the biologically active polypeptide or protein from an adenylate kinase.

12. The fusion protein of claim 1 wherein the biological active polypeptide or protein is a member of TNF family of cytokines.

13. The fusion protein of claim 12 wherein the member of TNF family of cytokines is TNFα.

14. The fusion protein of claim 13 having amino acid sequence identified by SEQ. ID 1.

15. The fusion protein of claim 13 having amino acid sequence identified by SEQ. ID 2.

16. The fusion protein of claim 12 wherein the member of TNF family of cytokines is 4-1BB-L.

17. The fusion protein of claim 16 having amino acid sequence identified by SEQ. ID 3.

18. The fusion protein of claim 16 having amino acid sequence identified by SEQ. ID 4.

19. The fusion protein of claim 12 wherein the member of TNF family of cytokines is RANK-L.

20. The fusion protein of claim 12 wherein the member of TNF family of cytokines is OX40L.

21. The fusion protein of claim 12 wherein the member of TNF family of cytokines is Trail.

22. The fusion protein of claim 6 wherein the biological active polypeptide or protein is a member of TNF family of cytokines.

23. The fusion protein of claim 22 wherein the member of TNF family of cytokines is TNFα.

24. The fusion protein of claim 23 having amino acid sequence identified by SEQ. ID 1.

25. The fusion protein of claim 23 having amino acid sequence identified by SEQ. ID 2.

26. The fusion protein of claim 22 wherein the member of TNF family of cytokines is 4-1BB-L.

27. The fusion protein of claim 26 having amino acid sequence identified by SEQ. ID 3.

28. The fusion protein of claim 26 having amino acid sequence identified by SEQ. ID 4.

29. The fusion protein of claim 22 wherein the member of TNF family of cytokines is RANK-L.

30. The fusion protein of claim 22 wherein the member of TNF family of cytokines is OX40L.

31. The fusion protein of claim 22 wherein the member of TNF family of cytokines is Trail.

32. The fusion protein of claim 1 wherein the biological active polypeptide or protein is T4 DNA Ligase.

33. The fusion protein of claim 33 having amino acid sequence identified by SEQ. ID 5.
